(19) United States
(12) Patent Application Publication
(10) Pub. No.: US 2024/0131315 A1
(43) Pub. Date: Apr. 25, 2024

(54) VASCULAR ACCESS ASSEMBLIES

(71) Applicant: Merit Medical Systems, Inc., South Jordan, UT (US)

(72) Inventors: John Hall, Bountiful, UT (US); Wayne Mower, Bountiful, UT (US); Christopher Cindrich, Highland, UT (US)

(21) Appl. No.: 18/488,402

(22) Filed: Oct. 16, 2023

Related U.S. Application Data
(60) Provisional application No. 63/380,339, filed on Oct. 20, 2022.

(51) Int. Cl. A61M 39/02

(52) U.S. Cl. CPC A61M 39/0247

(57) ABSTRACT

Vascular access assemblies for facilitating hemodialysis of a patient. The vascular access assembly may include a first tubular conduit and a bifurcated stent disposed in the superior vena cava. The vascular access assembly may further include a second tubular conduit that connects that couples an arm of the bifurcated stent to the first tubular conduit to create a blood flow pathway between two locations of a vasculature of a patient.

### RELATED APPLICATIONS

This application claims priority to U.S. Provisional Application No. 63/380,339, filed on Oct. 20, 2022 and titled, “Vascular Access Assemblies,” which is hereby incorporated by reference in its entirety.

### TECHNICAL FIELD

The present disclosure relates generally to the field of vascular access assemblies. More particularly, some embodiments relate to vascular access assemblies that facilitate hemodialysis.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a vascular access assembly according to one embodiment of the present disclosure implanted in a patient.

FIG. 2 illustrates an exploded view of a vascular access assembly according to one embodiment of the present disclosure.

FIG. 3 illustrates an exploded view of a vascular access assembly according to one embodiment of the present disclosure.

FIG. 4 illustrates an exploded view of a vascular access assembly according to one embodiment of the present disclosure.

FIG. 5 illustrates a vascular access assembly according to one embodiment of the present disclosure implanted in a patient.

### DETAILED DESCRIPTION

Many patients who suffer from kidney malfunction undergo hemodialysis to remove waste products from their blood. Hemodialysis generally requires access to an adequate blood supply. In some cases, access to a blood supply may be established via an arteriovenous fistula. In other circumstances, other methods for accessing the blood supply are used.

For example, in some embodiments, access to a blood supply is established via an arteriovenous graft. In other embodiments, access to a blood supply is established via a graft that extends from a peripheral blood supply to an outlet that is positioned in the central venous system.

Certain embodiments disclosed herein may be used to establish an artificial blood flow pathway between two locations of a vasculature of a patient, such as along a non-natural or artificial conduit, that improves or provides alternative access to a blood supply. The artificial flow path may be used, for example, to bypass a central venous stenosis. In some embodiments, the artificial blood flow pathway, when implanted into a patient, is fully subcutaneous. Access to a blood supply that is provided by an artificial flow path may be particularly advantageous for access in hemodialysis patients (such as hemodialysis patients who have exhausted peripheral venous access sites for fistulas).

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and fluidic interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

The terms “central” and “peripheral,” as used herein, are opposite directional terms along a flow path of the vasculature. For example, a peripheral end of a device or component is the end of the device or component that is furthest from the heart when the device or component is assembled and implanted within the patient. The central end refers to the opposite end or the end closest to the heart of the patient when the device is in use. Further, this reference frame is applied herein to devices configured or designed to have one end (a central end) positioned closer to the heart when the device is in use, whether or not the device itself is deployed within the body.

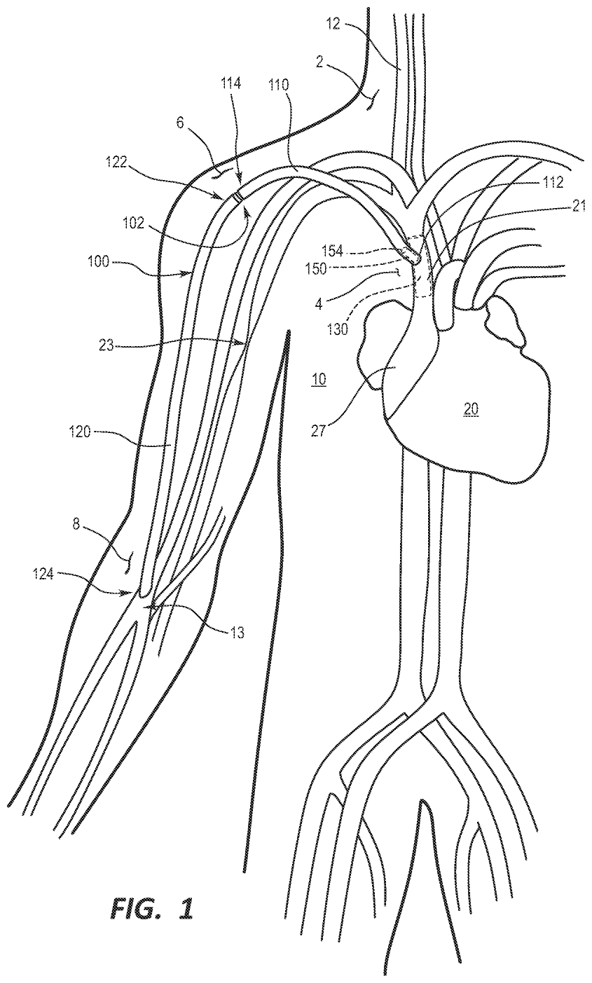

FIG. 1 illustrates a vascular access system 100 implanted in a patient 10 that may be used in hemodialysis patients who have exhausted peripheral venous access sites for fistulas. The illustrated vascular access system 100 is not limited to hemodialysis procedures and may be used in a number of other procedures or for different uses.

As discussed above, the vascular access assembly 100 may be used in any suitable medical procedure, such as to establish vascular access for hemodialysis. For example, where a vein has become stenotic or otherwise failed (e.g. central venous stenosis 23) an artificial flow path that bypasses the stenosis 23 or failure may be established. Stated another way, an artificial flow path may be established from a target site 13 (e.g., from a target site in a vessel, artery, arteriovenous graft, etc.) to the vena cava 21 or right atrium 27 of the heart 20. Various examples herein discuss access and therapies performed in the right atrium 27 of the heart 20. These examples and related disclosure may be analogously applied to access and therapies performed at adjacent locations such as the vena cava 21 or the venous vasculature around the vena cava 21.

As shown, the vascular access assembly 100 can include a first tubular conduit 110, a second tubular conduit 120, a bifurcated stent 130, and one or more connectors or adaptors 102. The terminology of first tubular conduit 110 and second tubular conduit 120 may be reversed, such that the second tubular conduit 120 is the first tubular conduit 120 and vice versa. As discussed above, the vascular access assembly 100 may bypass a central venous stenosis 23 or collapsed vein to provide an alternative flow path from a vein or artery to a heart 20 of the patient 10.

The bifurcated stent 130 of the vascular access assembly 100 is disposed in the superior vena cava 21. An arm 150 of the bifurcated stent 130 extends of out the superior vena cava 21 and coupes to a central end 112 of the first tubular conduit 110. In some embodiments, a peripheral end 154 of the arm 150 couples to a central end 112 of the first tubular conduit 110. A peripheral end 114 of the first tubular conduit 110 couples to a central end 122 of the second tubular conduit 120. In the illustrated embodiment, the central end 122 of the second tubular conduit 120 may be coupled to the peripheral end 114 of the first tubular conduit 110 by a connector 102. The peripheral end 124 of the second tubular conduit 120 may be coupled to an artery at the target site 13. For example, an anastomosis may be performed between the peripheral end 124 of the second tubular conduit 120 and a vessel at the target site 13.

The illustrated vascular access assembly 100 is disposed beneath the skin of the patient and is disposed outside of the patient's vasculature except for a portion of the bifurcated stent 130 that is disposed within the superior vena cava 21 near the right atrium 27 of the heart 20 of the patient 10. In other words, a majority of the vascular access assembly 100 is subcutaneous and external to the vasculature.

The implanted vascular access assembly 100 may be used to facilitate vascular access. For example, in the case of hemodialysis, a practitioner may insert a first needle through the skin of the patient 10 and the vascular access assembly 100. More particularly, the first needle may be inserted into the second tubular conduit 120. Fluid may be withdrawn from the vascular access assembly 100 and drawn into a dialysis machine that purifies the blood. The purified blood may then be returned to the patient 10 via a second needle that extend through the skin of the patient 10 into a more central location of the second tubular conduit 120.

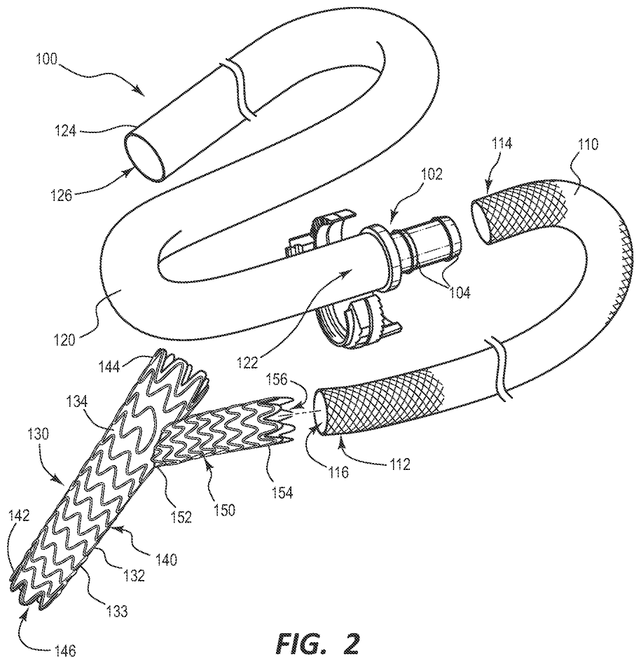

FIG. 2 illustrates an exploded view of the vascular access assembly 100. The vascular access assembly 100 includes the first tubular conduit 110, the second tubular conduit 120, the bifurcated stent 130, and the one or more connectors or adaptors 102. The first tubular conduit 110 comprises a central end 112, a peripheral end 114, and a lumen 116 that extends from the central end 112 to the peripheral end 114. In some embodiments, the first tubular conduit 110 may have an initial length of at least 20 cm, at least 25 cm, at least 30 cm, or at least 35 cm. For example, the first tubular conduit 210 may have an initial length of between about 20 cm and about 50 cm or between about 35 cm and about 45 cm. In certain embodiments, the first tubular conduit 110 has an internal diameter of between about 3.5 mm and about 6.5 mm. For example, the internal diameter of the first tubular conduit 210 may be between about 4.5 mm and about 5.5 mm. The length of the first tubular conduit 110 may be shortened so that the length of the first tubular conduit 110 is adequate for the placement of the vascular access assembly 100.

In various embodiments, the first tubular conduit 110 may be resistant to kinking and/or crush forces. The first tubular conduit 110 may be reinforced. For example, in the illustrated embodiment of FIG. 2, the first tubular conduit 110 is reinforced with nitinol, such as braided nitinol, which can provide resistance to kinking and/or crush forces. More specifically, in various embodiments, the first tubular conduit 110 may include silicone-coated nitinol.

In some embodiments, the first tubular conduit 110 may include one or more radiopaque bands or markers (not shown). For example, the first tubular conduit 110 may include a radiopaque band adjacent the central end 112 of the first tubular conduit 110. The radiopaque band(s) or marker(s) may facilitate fluoroscopic placement of the first tubular conduit 110 within the patient 10.

The second tubular conduit 120 comprises a central end 122, a peripheral end 124, and a lumen 126 that extends from the central end 122 to the peripheral end 124. In certain embodiments, the second tubular conduit 120 may be configured to be accessed for hemodialysis. In other words, during some medical procedures (e.g., hemodialysis), the second tubular conduit 120 may be accessed in lieu of the natural vasculature of a patient. In various embodiments, the second tubular conduit 120 may include and/or consist of polytetrafluoroethylene (PTFE) (e.g., such as expanded PTFE (ePTFE), rotational spun PTFE, or electrospun PTFE). In various other embodiments, the second tubular conduit 120 may include silicone, a fibrous polymer, or another suitable material.

In some embodiments, the second tubular conduit 120 may include a puncturable and self-sealing wall such that the wall may be punctured by insertion of a needle and then reseal upon withdrawal of the needle. The self-sealing wall may be of any suitable composition. In certain embodiments, the self-sealing wall may be a multi-layered construct. For example, the self-sealing wall may include an outer layer, an inner layer, and at least one tie layer disposed between the outer layer and the inner layer. One or more of the outer layer and the inner layer may include PTFE. For example, the outer layer may include and/or consist of expanded PTFE while the inner layer may include and/or consist of rotational spun or electrospun PTFE. The tie layer may include an elastomer such as elastomeric silicone. Due, at least in part, to the properties of the silicone, the resulting construct may be self-sealing. In other words, when a needle that has been inserted through the wall is withdrawn from the second tubular conduit 120, the wall may seal itself, thereby preventing leakage of blood from the second tubular conduit 120.

In various embodiments, the second tubular conduit 120 may have an initial length of at least 30 cm, at least 40 cm, or at least 45 cm. For example, the second tubular conduit 120 may be between about 30 cm and about 70 cm or between about 40 cm and about 60 cm in length. In some embodiments, the second tubular conduit 120 may have an internal diameter of between about 4.5 mm and about 8 mm. For example, the internal diameter of the second tubular conduit 120 may be between about 5.5 mm and about 6.5 mm. The length of the second tubular conduit 120 may be shortened so that the length of the second tubular conduit 120 is adequate for the placement of the vascular access assembly 100.

In some embodiments, both the first tubular conduit 110 and the second tubular conduit 120 may be self-sealing. In some other embodiments, only the second tubular conduit 120 may be self-sealing.

The bifurcated stent 130 comprises a body 140 and the arm 150. The body 140 includes a central end 142 and a peripheral end 144 and the arm 150 includes a central end 152 that coupled to the body 140 and a peripheral end 154. The body 140 includes a lumen 146 that extends from the central end 152 to the peripheral end 144. The arm 150 also includes a lumen 156 that extends from the central end 152 to the peripheral end 154 and is in communication with the lumen 146 of the body 140. In some embodiments, the arm 150 is integral with the body 140 and in some embodiments the arm 150 is coupled to the body 140. The arm 150 may extend away from the body 140 at an angle relative to the body 140. The angle may be between 15 degrees and 90 degrees.

The bifurcated stent 130 may include a scaffolding structure or frame 132 and a covering 134 disposed over at least a portion of the frame 132. In some embodiments, the frame 132 provides the structure for both the body 140 and the arm 150 of the bifurcated stent 130. The frame 132 is configured to resist radial compression when bifurcated stent 130 is disposed in a body lumen of the patient 10. As discussed above, the bifurcated stent 130 may be disposed in the superior vena cava 21. In some embodiments, the frame 132 may consist of a single continuous wire 133 forming a plurality of helixes that wrap around forming the lumens 146, 156 of both the body 140 and the arm 150. In some embodiments, the frame 132 may comprise more than one wire 133. The wire 133 may be comprised of Nitinol (ASTM F2063), or other suitable materials. In some embodiments, an area of the body 140 surrounding the central end 152 of the arm 150 may be void of wires for the structure or frame 132. In the void area as seen in FIG. 2, a zig-zag pattern of the wire frame 132 may loop back on itself to prevent the wire frame 132 from crossing over the junction between the body 140 and the arm 150.

In the illustrated embodiment of FIG. 2, the wire 133 may be shaped in a wave-type configuration, the waves defining apexes and arms of the frame 132. The length of each arm of the wire 133 may vary in length and may vary in length along a longitudinal length of the frame 132 itself. The apexes are longitudinally separated along the longitudinal length of the body bifurcated stent 130. Along some portions of the bifurcated stent 130, the adjacent helixes of the wire 133 in the longitudinally direction are evenly spaced. Along other portions of the bifurcated stent 130, the space between adjacent helixes in the longitudinal direction are not evenly spaced. Again, as noted above, the wire 133 may loop back on itself to create a portion of the body 140 where the arm 150 can connect without the wire 133 covering the junction. The wire 133 may have a variety of different shapes and sizes to form the frame 132 to support the bifurcated stent 130. Additionally, the frame 132 may be configured to allow the entire bifurcated stent 130 to be crimped into a relatively low-profile configuration for delivery

As discussed above, the bifurcated stent 130 comprises the covering 134 that at least partially covers the frame 132. In the illustrated embodiment of FIG. 2, an outer portion of the frame 132 is visible and an inner portion of the frame 132 may be encompassed in the covering 134.

In certain embodiments, one or both of an inner surface and an outer surface of the vascular access assembly 100 may be associated with a therapeutic agent. In other words, the therapeutic agent may be disposed on or embedded within a surface of the vascular access assembly 100. The therapeutic agent may be released from the surface(s) of the vascular access assembly 100 to deliver a therapeutically effective dose of the therapeutic agent to the patient when the vascular access assembly 100 is implanted within the patient 10. In various embodiments, a first therapeutic agent is associated with the inner surface of the vascular access assembly 100 and a second therapeutic agent that differs from the first therapeutic agent is associated with the outer surface of the vascular access assembly 100. In such embodiments, both the first therapeutic agent and the second therapeutic agent may be delivered into the bloodstream of the patient in therapeutically effective doses when the vascular access assembly 100 is implanted within the patient. In some embodiments, heparin may be used as a therapeutic agent. The therapeutic agent may reduce or be configured to reduce thrombus or tissue proliferation.

With continued reference to FIG. 2, the one or more connectors 102 may facilitate coupling of the first tubular conduit 110 to the second tubular conduit 120, or vice versa. In certain embodiments, such as the embodiment shown in FIG. 2, the connector 102 can be disposed at a central end 122 the second tubular conduit 120.

As depicted, the connector 102 may include one or more barbs or protrusions 104 that are designed to engage with an inner surface of the first tubular conduit 110 to form a fluid-tight connection. While FIG. 2 shows the connector 102 at the central end 122 of the second tubular conduit 120, a skilled artisan will recognize that, in other embodiments, the connector 102 may instead be disposed at a peripheral end 114 of the first tubular conduit 120. In still other embodiments, the connector 102 may include components disposed at both the central end 122 of the second tubular conduit 120 and the peripheral end 114 of the first tubular conduit 110. The connector 102 may be made from any suitable material, such as a metal (e.g., steel or titanium), a polymer, etc.

The body 140 of the bifurcated stent 130 may be radially expanded (e.g., self-expanded or balloon expanded) to compress the body 140 against the superior vena cava 21 to secure the body 140 of the bifurcated stent 130 in place.

The arm 150 of the bifurcated stent 130 may be radially expanded (e.g., self-expanded or balloon expanded) to couple the arm 150 to the first tubular conduit 110. The arm 150 may be inserted into the lumen 116 of the central end 112 of the first tubular conduit 110 and the arm 150 may be expanded to compress the arm 150 against the inner surface of the first tubular conduit 110 to secure the arm 150 to the first tubular conduit 110. In some embodiments, a majority of the arm 150 may be inserted into the first tubular conduit 110. In some embodiment, only a portion of the arm 150, such as the peripheral end 154 of the arm 150 is inserted into the first tubular conduit 110. The coupling between the arm 150 of the bifurcated stent 130 and the first tubular conduit 110 creates a seal to prevent leakage between the arm 150 of the bifurcated stent 130 and the first tubular conduit 110.

In some embodiment, the first tubular conduit 110 may be coupled to the arm 150 of the bifurcated stent 130 by a separate connector 102. The coupling between the arm 150 of the bifurcated stent 130 and the first tubular conduit 110 creates a seal to prevent leakage between the arm 150 of the bifurcated stent 130 and the first tubular conduit 110.

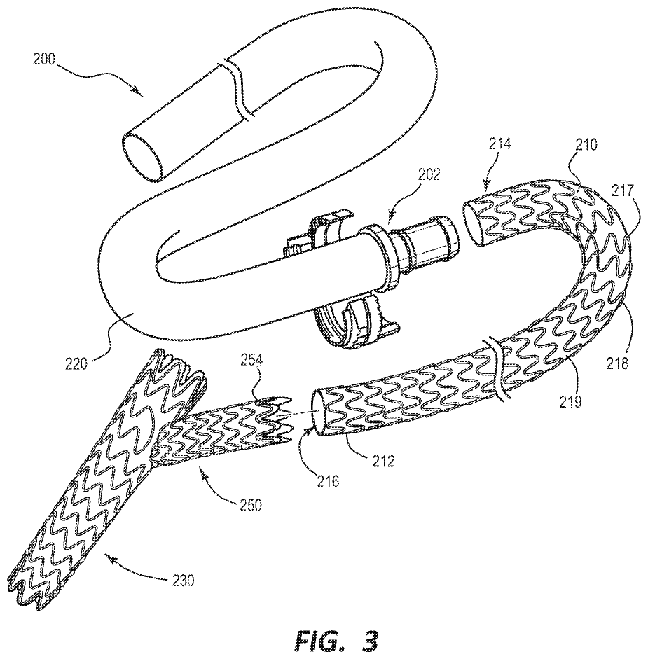

FIG. 3 depicts an embodiment of a vascular access assembly 200 that resembles the vascular access assembly 100 described above in certain respects. Accordingly, like features are designated with like reference numerals, with the leading digits incremented to “2.” For example, the embodiment depicted in FIG. 3 includes a first tubular conduit 210 that may, in some respects, resemble the first tubular conduit 110 of FIGS. 1 and 2. Relevant disclosure set forth above regarding similarly identified features thus may not be repeated hereafter. Moreover, specific features of vascular access assembly 100 and related components shown in FIGS. 1 and 2 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the vascular access assembly 200 and related components depicted in FIG. 3. Any suitable combination of the features, and variations of the same, described with respect to the vascular access assembly 100 and related components illustrated in FIGS. 1 and 2 can be employed with the vascular access assembly 200 and related components of FIG. 1, and vice versa. This pattern of disclosure applies equally to further embodiments depicted in subsequent figures and described hereafter, wherein the leading digits may be further incremented.

The vascular access assembly 200 includes a first tubular conduit 210, a second tubular conduit 220, a bifurcated stent 230, and one or more connectors or adaptors 202. The first tubular conduit 210 comprises a central end 212, a peripheral end 214, and a lumen 216 that extends from the central end 212 to the peripheral end 214.

The first tubular conduit 210 may be radially expanded (e.g., self-expanded or balloon expanded). In various embodiments, the first tubular conduit 210 may be resistant to kinking and/or crush forces. The first tubular conduit 210 may be reinforced with a frame 217, which can provide resistance to kinking and/or crush forces.

The first tubular conduit 210 may further include a covering 219 disposed over at least a portion of the frame 217. In some embodiments, the frame 217 provides the structure for the first tubular conduit 210. In some embodiments, the frame 217 may consist of a single continuous wire 218 forming a plurality of helixes that wrap around forming the lumen 216. In some embodiments, the frame 217 may comprise more than one wire 218. The wire 218 may be comprised of Nitinol (ASTM F2063), or other suitable materials. In the illustrated embodiment of FIG. 3, the wire 218 may be shaped in a wave-type configuration, the waves defining apexes and arms of the frame 217.

As discussed above, the first tubular conduit 210 comprises the covering 219 that at least partially covers the frame 217. In the illustrated embodiment of FIG. 3, an outer portion of the frame 217 is visible and an inner portion of the frame 217 may be encompassed in the covering 219. In some embodiments, the covering 219 of the first tubular conduit 210 may be silicone.

The second tubular conduit 220, the bifurcated stent 230, and the connector 202 are similar to the second tubular conduit 120, the bifurcated stent 130, and the connector 102 are similar to the illustrated embodiment of FIGS. 1 and 2.

The first tubular conduit 210 and the bifurcated stent 230 may couple to each other in a variety of different ways. For example, an arm 250 of the bifurcated stent 230 may be inserted into the lumen 216 of central end 212 of the first tubular conduit 210 and the arm 250 may be expanded to compress the arm 250 against the inner surface of the first tubular conduit 210 to secure the arm 250 to the first tubular conduit 210. The coupling between the arm 250 of the bifurcated stent 230 and the first tubular conduit 210 creates a seal to prevent leakage between the arm 250 of the bifurcated stent 230 and the first tubular conduit 210. In some embodiments, a majority of the arm 250 may be inserted into the first tubular conduit 210. In some embodiment, only a portion of the arm 250, such as the peripheral end 254 of the arm 250 is inserted into the first tubular conduit 210. In this situation, the arm 250 may expand to a wider diameter than the first tubular conduit 210 to ensure the seal.

In some embodiments, the central end 212 of the first tubular conduit 210 may be inserted in the peripheral end 254 of the arm 250 and the first tubular conduit 210 may be expanded to compress the first tubular conduit 210 against the inner surface of the arm 250 to secure the first tubular conduit 210 to the arm 250 of the bifurcated stent 230. The coupling between the arm 250 of the bifurcated stent 230 and the first tubular conduit 210 creates a seal to prevent leakage between the arm 250 of the bifurcated stent 230 and the first tubular conduit 210. In this situation, the first tubular conduit 210 may expand to a wider diameter than the arm 250 to ensure the seal.

In some embodiments, the first tubular conduit 210 may be coupled to the arm 250 of the bifurcated stent 230 by a separate connector 202. The coupling between the arm 250 of the bifurcated stent 230 and the first tubular conduit 210 creates a seal to prevent leakage between the arm 250 of the bifurcated stent 230 and the first tubular conduit 210.

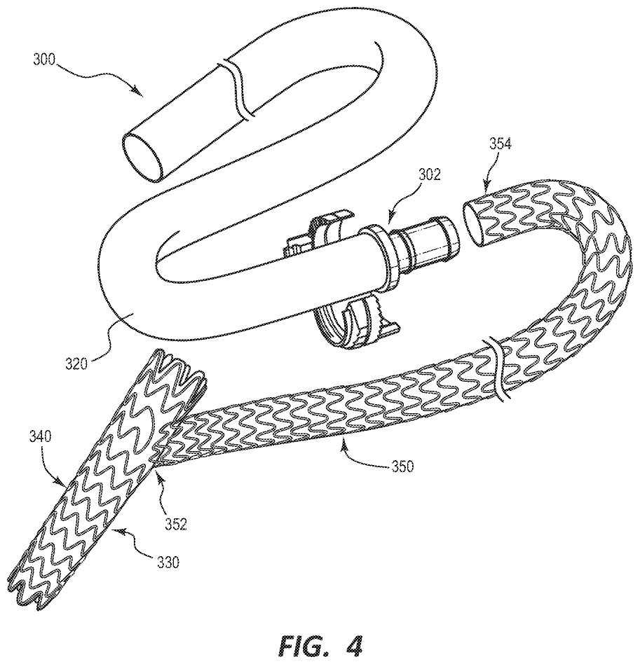

FIG. 4 depicts an embodiment of a vascular access assembly 300. The vascular access assembly 300 includes a tubular conduit 320, a bifurcated stent 330 with an arm 350, and one or more connectors or adaptors 302. The tubular conduit 320 is similar to the second tubular conduit 120 of the illustrated embodiment of FIG. 2. The connector 302 is similar to the connector 102 of the illustrated embodiment of FIG. 2. The bifurcated stent 330 is similar to the bifurcated stent 130 of the illustrated embodiment of FIG. 2 with a variation to a length of an arm 150 of the bifurcated stent 130. In the illustrated embodiment, of FIG. 4, the length of the arm 350 is longer and extends from a body 340 of the bifurcated stent 330 to the tubular conduit 320. In other words, the arm 350 of the bifurcated stent 330 essentially takes the place of the first tubular conduit 110.

The arm 350 of the bifurcated stent 330 has a central end 352 that is coupled to or integral with the body 340 and a peripheral end 354 that is coupled to the tubular conduit 320. The connector 302 may facilitate coupling of the tubular conduit 320 to the arm 350 of the bifurcated stent 330, or vice versa. In certain embodiments, such as the embodiment shown in FIG. 4, the connector 302 can be disposed at a central end 322 the tubular conduit 320.

The body 340 and the arm 350 of the bifurcated stent 330 may be radially expanded (e.g., self-expanded or balloon expanded) to compress the body 340 against the superior vena cava 21 to secure the body 340 of the bifurcated stent 330 in place.

Referring back to FIG. 1, various incisions 2, 4, 6, 8, and be made during a medical procedure to properly place the vascular access assembly 100. The first incision 2 may enable medical professional to advance the bifurcated stent 130 through the right internal jugular vein 12 to the superior vena cava 21 near the right atrium 27 of the heart 20.

The second incision 4 may enable the medical professional to open the superior vena cava 21 to allow the arm 150 of the bifurcated stent 130 to extend out of the superior vena cava 21 so that the arm 150 may couple to the first tubular conduit 110. In some embodiments, an anastomosis may also be performed between the first tubular conduit 110 and the superior vena cava 21. In some embodiments, this incision is not necessary.

A third incision 6 may be made in the shoulder region of the patient 10 (e.g. adjacent to the deltopectoral groove). A tunneling device may be used to establish a subcutaneous path between the second incision 4 near the superior vena cava 21 and a third incision 6 in the shoulder region of the patient 10. The peripheral end 114 of the first tubular conduit 110 may be inserted into the second incision 4 and advanced along the path established by the tunneling device (i.e., the first tubular conduit 110 is tunneled) such that the first tubular conduit 110 extends from the arm 150 of the bifurcated stent 130 to the third incision 6 in the should region of the patient 10.

A fourth incision 8 may be made near the target site 13. In some other embodiments, the target site 13 may be an arteriovenous graft, a vein, or another suitable position. A tunneling device may then be used to establish a subcutaneous path between the third incision 6 in the shoulder region of the patient 10 to the target site 13. A peripheral end 124 of the second tubular conduit 120 may then be inserted into the third incision 6 and advanced along the path established by the tunneling device (i.e., the second tubular conduit 120 is tunneled) such that the second tubular conduit 120 extends from the third incision 6 in the shoulder region of the patient 50 to the target site 13.

With the central end 112 of the first tubular conduit 110 coupled to the arm 150 of the bifurcated stent 130, the peripheral end 114 of the first tubular conduit 110 may then, if needed, be cut to the appropriate length. In other words, the first tubular conduit 110 may initially (e.g., when manufactured and inserted as described above) have a length that is longer than is needed to establish a flow path from the superior vena cava 21 of the heart 20 of the patient 10 to the second incision 4 in the shoulder region of the patient 10. The first tubular conduit 110 may then be cut to proper length to facilitate coupling of the second tubular conduit 120 to the first tubular conduit 110 at the second incision 4 in the shoulder region of the patient 10. As depicted, the central end 122 of the second tubular conduit 120 may be coupled to the peripheral end 114 of the first tubular conduit 110 via the connector 102.

Similarly, in some embodiments, the second tubular conduit 120 may have an initial length that is longer than is needed to establish a flow path from the second incision 4 in the shoulder region of the patient 10 to the target site 13. In such embodiments, the central end 122 of the second tubular conduit 120 may be cut to the appropriate length once the second tubular conduit 120 has been inserted into the patient 10. In some embodiments, the connector 102 may then be attached to the newly formed central end 122 of the second tubular conduit 120. In some other embodiments, no cutting of the second tubular conduit 120 may be needed.

Once the first tubular conduit 110 and the second tubular conduit 120 are the proper length, the second tubular conduit 120 may be coupled to the first tubular conduit 110, or vice versa. For example, the connector 102 at the central end 122 of the second tubular conduit 120 may be inserted into the peripheral end 114 of the first tubular conduit 110 such that the barbs or protrusions 104 of the connector 102 engage with an inner surface of the first tubular conduit 110 (see FIG. 2). Such engagement may establish a fluid-tight connection between the first tubular conduit 110 and the second tubular conduit 120. Establishment of a fluid-tight connection can be confirmed by attaching the peripheral end 128 of the second tubular conduit 120 to a syringe and advancing fluid (e.g., heparinized saline) through the system.

The peripheral end 124 of the second tubular conduit 120 may be coupled to an artery at the target site 13. For example, an arterial anastomosis may be performed between the peripheral end 124 of the second tubular conduit 120 and a vessel at the target site 13. Coupling of a portion of the vascular access assembly 100 (e.g., the peripheral end 124 of the second tubular conduit 120) to an artery may be performed via any suitable technique. Once a flow path from the target site 13 to the heart 20 has been established as shown in FIG. 1, the first incision 2, the second incision 4, third incision 6, and the fourth incision 8 may be closed via any suitable technique. In this manner, the vascular access assembly 100 may, when implanted and assembled, be a fully subcutaneous surgical implant. Furthermore, the implanted and assembled vascular access assembly 100 may, as described above, be implanted without establishing a venous anastomosis.

The steps of the procedure described above are only exemplary in nature. In other words, the vascular access assembly 100 may be implanted into the patient 10 via a procedure that deviates somewhat from the procedure described above. One of ordinary skill in the art, having the benefit of this disclosure, will also appreciate that some of the steps described above need not be performed in the order that is specified above.

An additional aspect of the disclosure relates to methods of accessing an implanted vascular access assembly 100. A practitioner may desire to access the vascular access assembly 100 so that the practitioner may clean or clear at least a portion of the vascular access assembly 100. In some embodiments, the vascular access assembly 100, or at least a portion of the vascular access assembly 100, may become occluded and/or blocked during use. For example, a blood clot or other embolus may develop within at least a portion of the vascular access assembly 100. Accordingly, the practitioner may access the vascular access assembly 100 to remove the blood clot or other embolus from within the vascular access assembly 100. The methods may also be used and/or adapted for the removal of other types of emboli from within the vascular access assembly 100 (e.g., fatty deposits, tissue growths, etc.).

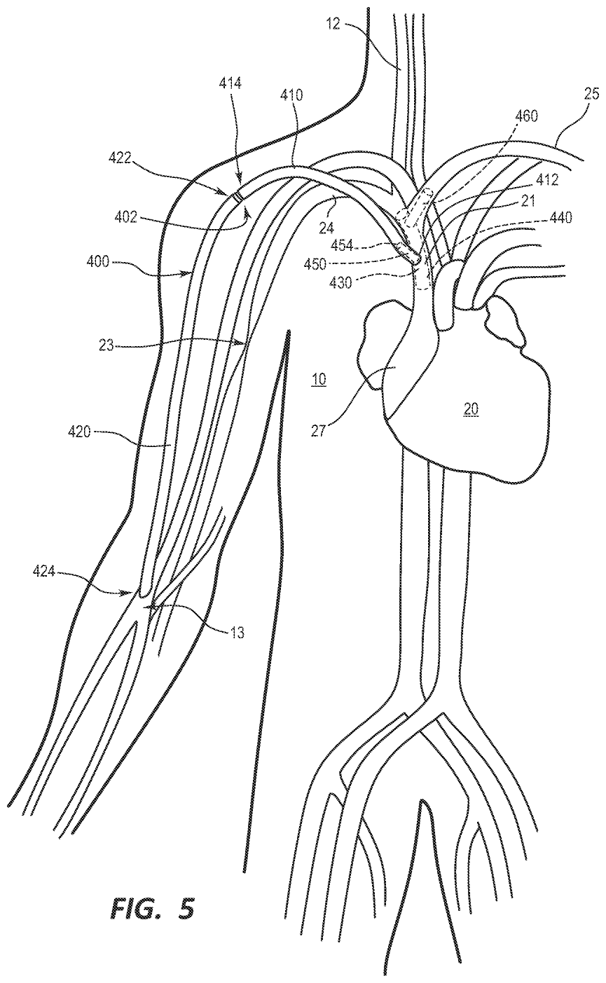

FIG. 5 illustrates a vascular access system 400 implanted in the patient 10 that may be used in hemodialysis patients who have exhausted peripheral venous access sites for fistulas. Similar to the vascular access system 100 of FIG. 1, the vascular access system 400 may include a first tubular conduit 410, a second tubular conduit 420, a bifurcated stent 430, and one or more connectors or adaptors 402. The vascular access assembly 400 may bypass a central venous stenosis 23 or collapsed vein to provide an alternative flow path from a vein or artery to a heart 20 of the patient 10.

The bifurcated stent 430 of the vascular access assembly 400 is disposed in the superior vena cava 21. An arm 450 of the bifurcated stent 430 extends of out the superior vena cava 21 and coupes to a central end 412 of the first tubular conduit 410. In some embodiments, a peripheral end 434 of the arm 450 couples to a central end 412 of the first tubular conduit 410. A peripheral end 414 of the first tubular conduit 410 couples to a central end 422 of the second tubular conduit 420. In the illustrated embodiment, the central end 422 of the second tubular conduit 420 may be coupled to the peripheral end 414 of the first tubular conduit 410 by a connector 402. The peripheral end 424 of the second tubular conduit 420 may be coupled to an artery at a target site 13. For example, an anastomosis may be performed between the peripheral end 424 of the second tubular conduit 420 and a vessel at the target site 13.

In the illustrated embodiment, a body 440 of the bifurcated stent 430 may extend out of the superior vena cava 21. For example, the peripheral end 444 of the body 440 of the bifurcated stent 430 may be at the base right internal jugular vein 12 and the right subclavian vein 24. The bifurcated stent 430 may also include a plurality of arms to extend into veins adjacent to the birfurcated stent 430. For example, the birfurcated stent 430 may further include a second arm 460 that extends into the left brachiocefalic vein 25.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A vascular access assembly for provide a blood flow pathway between two locations of a vasculature of a patient comprising:
a bifurcated stent comprising a body and an arm, the body of the bifurcated stent is disposed within a superior vena cava of the patient and the arm of the bifurcated stent extends out of the superior vena cava external to the vasculature of the patient; and
a first tubular conduit comprising a peripheral end, a central end, and a lumen,
wherein the peripheral end of the first tubular conduit is configured to attached to a vessel of the patient, and
wherein the first tubular conduit and the bifurcated stent form a blood flow pathway between the vessel and the superior vena cava of the patient.

2. The vascular access assembly of claim 1, wherein the first tubular conduit is external to the vasculature of the patient.

3. The vascular access assembly of claim 1, wherein the peripheral end of the first tubular conduit is coupled to an artery in an arm of the patient by an anastomosis.

4. The vascular access assembly of claim 1, wherein the bifurcated stent is an expandable stent.

5. The vascular access assembly of claim 4, wherein at least a peripheral end of the arm of the bifurcated stent extends into the central end of the first tubular conduit and expands to create a seal between the first tubular conduit and the arm of the bifurcated stent.

6. The vascular access assembly of claim 1, further comprising a connector configured to couple the first tubular conduit to the arm of the bifurcated stent.

7. The vascular access assembly of claim 1, wherein the first tubular conduit couples to the arm of the bifurcated stent in a shoulder region of the patient.

8. The vascular access assembly of claim 1, further comprising a second tubular conduit comprising a peripheral end, a central end, and a lumen,
wherein the central end of the first tubular conduit couples to the peripheral end of the second tubular conduit and the central end of the second tubular conduit is coupled to a peripheral end of the arm of the bifurcated stent.

9. The vascular access assembly of claim 8, wherein the first tubular conduit and the second tubular conduit are external to the vasculature of the patient.

10. The vascular access assembly of claim 8, wherein the second tubular conduit is reinforced to be resistant to kinking and/or crush forces.

11. The vascular access assembly of claim 8, wherein the second tubular conduit is reinforced with a braided nitinol.

12. The vascular access assembly of claim 8, wherein at least the peripheral end of the arm of the bifurcated stent extends into the central end of the second tubular conduit and expands to create a seal between the second tubular conduit and the arm of the bifurcated stent.

13. The vascular access assembly of claim 8, wherein the second tubular conduit is an expandable stent.

14. The vascular access assembly of claim 13, wherein the peripheral end of the second tubular conduit extends into the central end of the first tubular conduit and expands to create a seal between the first tubular conduit and the second tubular conduit.

15. The vascular access assembly of claim 8, wherein the first tubular conduit and the second tubular conduit are self-sealing.

16. The vascular access assembly of claim 1, wherein the bifurcated stent comprises more than one arm.

17. The vascular access assembly of claim 1, wherein the body comprises a wire frame with an opening in the wire frame at the junction between the body and the arm.

18. A method of assembling a vascular access assembly in a patient, comprising:
disposing a bifurcated stent within a superior vena cava of a patient, the bifurcated stent comprising a body and an arm that extends away from the body;
extending the arm of the bifurcated stent out of the superior vena cava so that the arm is external to the superior vena cava;
coupling a peripheral end of a first tubular conduit to a vessel of the patient; and
creating a flow path from the peripheral end of the first tubular conduit to the arm of the bifurcated stent.

19. The method of claim 18, further comprising expanding the body and the arm of the bifurcated stent.

20. The method of claim 18, further comprising coupling a peripheral end of the arm of the bifurcated stent to a central end of the first tubular conduit.

21. The method of claim 18, further comprising:
coupling a central end of the first tubular conduit to a peripheral end of a second tubular conduit; and
coupling a central end of the second tubular conduit to a peripheral end of the arm of the bifurcated stent.
